(19) United States
(12) Patent Application Publication
(10) Pub. No.: US 2015/0265341 A1
(43) Pub. Date: Sep. 24, 2015

(54) ELECTROPHYSIOLOGY SYSTEM

(71) Applicant: BOSTON SCIENTIFIC SCIMED, INC., MAPLE GROVE, MN (US)

(72) Inventor: JOSEF V. KOBLISH, SUNNYVALE, CA (US)

(73) Assignee: BOSTON SCIENTIFIC SCIMED, INC., MAPLE GROVE, MN (US)

(21) Appl. No.: 14/661,911

(22) Filed: Mar. 18, 2015

Related U.S. Application Data
(60) Provisional application No. 61/955,090, filed on Mar. 18, 2014.

(51) Int. Cl. A61B 18/14; A61B 5/042; A61B 5/0452

(52) U.S. Cl. CPC A61B 18/1492; A61B 5/0452; A61B 5/0422; A61B 2018/00351

(57) ABSTRACT

Radio frequency (RF) ablation systems and methods for using the radio frequency ablation systems are disclosed. The RF ablation system may include an elongated member, an RF generator, and a processor. The elongated member may include a distal portion including one or more electrodes and the RF generator may be operatively coupled to one or more of the electrodes. The processor, which may be coupled to one or more of the electrodes, may obtain an output signal from the electrodes and may monitor changes in an elevation of an ST segment of one or more electrogram (EGM) readings of the obtained output signal. The processor may determine a level of one or more characteristics that are proportional to an elevated ST segment of EGM.

### CROSS-REFERENCE TO RELATED APPLICATIONS

This application claims priority under 35 U.S.C. §119 to U.S. Provisional Application Ser. No. 61/955,090, filed Mar. 18, 2014, the entirety of which is incorporated herein by reference.

### TECHNICAL FIELD

The disclosure is directed to systems and methods for providing therapies. More particularly, the disclosure is directed to systems and methods for mapping and ablating cardiac tissue.

### BACKGROUND

Aberrant conductive pathways disrupt the normal path of the heart's electrical impulses. For example, conduction blocks can cause the electrical impulses to degenerate into several circular wavelets that disrupt the normal activation of the atria or ventricles. The aberrant conductive pathways create abnormal, irregular, and sometimes life-threatening heart rhythms called arrhythmias. Ablation is one way of treating arrhythmias and restoring normal contraction. The sources of the aberrant pathways (called focal arrhythmia substrates) are located or mapped using mapping electrodes situated in a desired location. After mapping, the physician may ablate the aberrant tissue. In radio frequency (RF) ablation, RF energy is directed from the ablation electrode through tissue to an electrode to ablate the tissue and form a lesion.

### SUMMARY

The present disclosure relates generally to systems and methods for providing therapies and for performing analyses while providing the therapies. It is contemplated that the analyses may include determining levels of contact force between an elongated member and tissue, orientation of the elongated member, and contact between the elongated member and tissue.

Accordingly, in one illustrative instance, a system may include an elongated member, a radio frequency generator, and a processor. The elongated member may have a distal portion that includes one or more electrodes. The radio frequency generator may be operatively coupled to one or more of the electrodes to generate energy that may be conveyed to one or more of the coupled electrodes. The processor may be operatively coupled to one or more of the electrodes and may be capable of obtaining output signals from one or more of the electrodes, where one or more of the output signals include an electrogram (EGM) reading. In some instances, the processor may be capable of monitoring an elevation of an ST segment of one or more of the EGM readings.

In another illustrative instance, a method may include positioning a distal portion of an elongated member at a location proximate a target tissue and obtaining output signals from each of the bipolar microelectrode pairs, where one or more of the output signals comprises an EGM reading. Elevations of ST segments of the obtained EGM readings may be monitored. In some cases, the positioned distal portion of the elongated member may include a tissue ablation electrode capable of applying ablation energy to the target tissue and a plurality of microelectrodes distributed about the tissue ablation electrode and electrically isolated therefrom, the plurality of microelectrodes defining a plurality of bipolar microelectrode pairs, each bipolar microelectrode pair capable of generating an output signal.

In another illustrative instance, a system may include an elongated member, a radio frequency generator, a mapping processor, and an indicator. The elongated member may have a distal portion that includes a tissue ablation electrode capable of applying ablation energy to the target tissue and a plurality of microelectrodes distributed about the tissue ablation electrode and electrically isolated therefrom, the plurality of microelectrodes defining a plurality of bipolar microelectrode pairs, each bipolar microelectrode pair is capable of generating an output signal. The radio frequency generator may be operatively coupled to the tissue ablation electrode to generate energy to be conveyed to the tissue ablation electrode. The processor may be operatively coupled to one or more of the electrodes and may be capable of obtaining output signals from one or more of the electrodes, where one or more of the output signals include an EGM reading from one of the bipolar microelectrode pairs. In some cases, an elevation of an ST segment of the EGM reading from a bipolar microelectrode pair may be compared by the mapping processor to elevations of ST segments of the EGM readings of the other bipolar microelectrode pairs to, at least in part, determine a level of contact force between the distal portion of the elongated member and the target tissue. The indicator may indicate the level of contact force between the distal portion of the elongated member and the target tissue.

The above summary is not intended to describe each embodiment or every implementation of the present disclosure. Advantages and attainments, together with a more complete understanding of the disclosure, will become apparent and will be appreciated by referring to the following description and claims taken in conjunction with the accompanying drawings.

### BRIEF DESCRIPTION OF THE DRAWINGS

The aspects of the disclosure may be more completely understood in consideration of the following detailed description of various embodiments in connection with the accompanying drawings, in which:

FIG. 1 is a schematic illustration of a radio frequency (RF) ablation system that may be used in accordance with various examples of the present disclosure;

FIG. 2 is a schematic illustration showing a conventional ablation catheter on the left and an embodiment of an RF ablation catheter of the present disclosure on the right;

FIG. 3A is a schematic illustration of a P-QRS-T wave of an electrogram (EGM) reading;

FIG. 3B is a schematic illustration of a P-QRS-T wave of an EGM reading having an elevated ST segment;

FIG. 4 is a schematic illustration of electrical signals obtained from electrodes of an RF ablation system that may be used in accordance with various examples of the present disclosure;

FIG. 5 is a schematic illustration of electrical signals obtained from electrodes of an RF ablation system that may be used in accordance with various examples of the present disclosure; and

FIG. 6 is a schematic flow diagram of a method of using an RF ablation system that may be used in accordance with various examples of the present disclosure.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Any relative terms, such as first, second, third, right, left, bottom, top, etc., used herein in connection with a feature are just that and are not meant to be limiting other than to be indicative of the relative relationship of the modified feature with respect to another feature.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative instances and are not intended to limit the scope of the disclosure. Selected features of any illustrative instance may be incorporated into an additional instance unless clearly stated to the contrary.

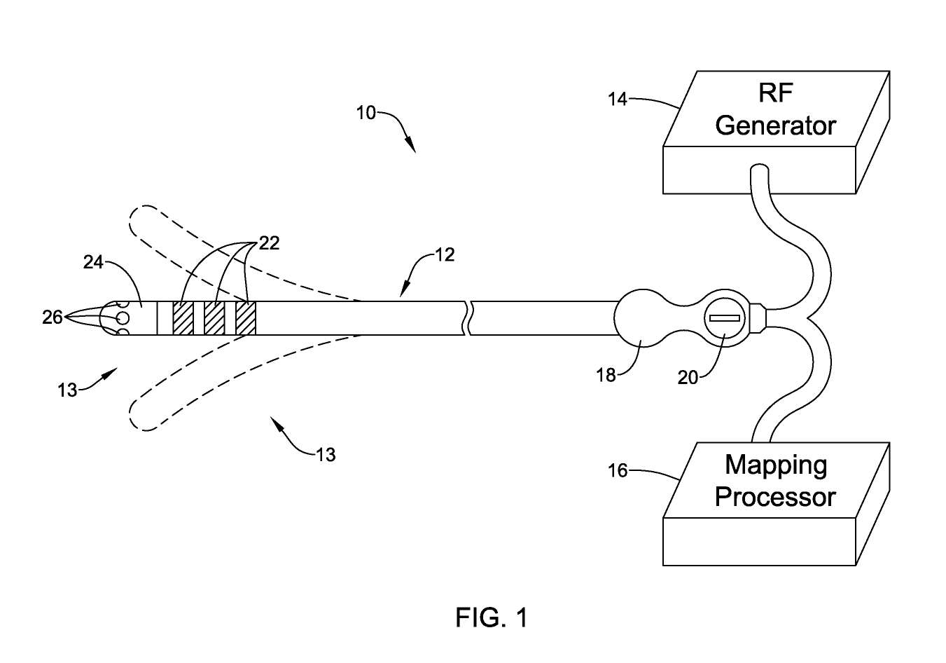

FIG. 1 is an illustrative radio frequency (RF) ablation system 10. As shown in FIG. 1, the system 10 may include one or more of an elongated member 12 (e.g., a catheter, ablation catheter, mapping catheter, or other elongated member), an RF generator 14, and a processor 16 (e.g., one or more of a mapping processor or other processor). Illustratively, the elongated member 12 may be operatively coupled to one or more (e.g., one or both) of the RF generator 14 and the processor 16. In some instances, the RF ablation system 10 may include an indicator in communication with the elongated member 12, the RF generator 14, and/or the processor 16. The indicator may be capable of indicating one or more characteristics or measures related to electrical signals sensed by the RF ablation system 10 (e.g., one or more characteristics related to or proportional to sensed or monitored elevations of ST segments, as discussed below, or other characteristics). The RF ablation system 10 may include one or more other features, as desired. In one instance, the RF ablation system 10 may include noise artifact isolators (not shown), where the microelectrodes 26 may be electrically insulated from the exterior wall by the noise artifact isolators

Illustratively, the RF generator 14 may be coupled to one or more electrodes of the elongated member 12. The coupling between the RF generator and one or more of the electrodes may be capable of facilitating conveyance of RF energy generated by the RF generator 14 to the coupled electrodes

The elongated member 12 may include a handle 18, which may have an actuator 20 (e.g., a control knob or other actuator). The handle 18 (e.g., a proximal handle) may be positioned at a proximal end of the elongated member 12 or at any other position along the elongated member 12. In some instances, the elongated member 12 may include a flexible body having a distal portion which may include one or more of a plurality of ring electrodes 22, tissue ablation electrodes 24 (e.g., tissue ablation electrodes or other electrodes), and microelectrodes 26 (e.g., mapping microelectrodes, which may be referred to as pin electrodes, or other microelectrodes) disposed or otherwise positioned within and/or electrically isolated from the ablation electrode 24, where one or more features of the distal end 13 of the elongated member 12 may be at least partially controlled through the handle 18.

In some instances, the RF ablation system 10 may be utilized in ablation procedures on a patient. As such, the elongated member 12 may be configured to be introduced through vasculature of a patient. Illustratively, the elongated member 12 may be inserted through the vasculature of the patient and into one or more chambers of the patient's heart or to one or more other target areas. When in the patient's vasculature or heart, the elongated member 12 may be used to map and/or ablate myocardial tissue using the microelectrodes 26 and/or the tissue ablation electrodes 24. In some instances, the ablation electrode 24 may be configured to apply ablation energy to myocardial tissue of the heart of a patient.

In some cases, the elongated member 12 may be steerable to facilitate navigating the vasculature of a patient or navigating other lumens. Illustratively, a distal portion of the elongated member 12 may be deflected by manipulation of the actuator 20 to effect steering of the elongated member 12. For example, the distal portion 13 of the elongated member 12 may be deflected by manipulation of the actuator 20 or in any other manner to position the tissue ablation electrodes 24 and/or the microelectrodes 26 adjacent target tissue. Additionally, or alternatively, the distal portion 13 of the elongated member 12 may have a pre-formed shape adapted to facilitate positioning the ablation electrode 24 and/or the microelectrodes 26 adjacent a target tissue. Illustratively, the preformed shape of the distal portion 13 of the elongated member 12 may be a radiused shape (e.g., a generally circular shape, a generally semi-circular shape) and/or may be oriented in a plane transverse to a general longitudinal direction of the elongated member 12.

The microelectrodes 26 may be circumferentially distributed about the ablation electrode 24 and/or electrically isolated therefrom. The microelectrodes 26 may be capable of operating or configured to operate in unipolar or bipolar sensing modes. In some instances, the plurality of microelectrodes 26 may define and/or at least partially form one or more bipolar microelectrode pairs 28.

In an illustrative instance, the elongated member 12 may have three microelectrodes 26 (e.g., a first microelectrode 26a, a second microelectrode 26b, and a third microelectrode 26c) distributed about the circumference of the tissue ablation electrode 24, such that the circumferentially spaced microelectrodes may form respective bipolar microelectrode pairs 28 of adjacent microelectrodes 26 (e.g., a first bipolar microelectrode pair 28a of the first microelectrode 26a and the second microelectrode 26b, a second bipolar microelectrode pair 28b of the second microelectrode 26b and the third microelectrode 26c, and a third bipolar microelectrode pair 28c of the third microelectrode 26c and the first microelectrode 26a). Each bipolar microelectrode pair 28 may be capable of generating, or may be configured to generate, an output signal corresponding to a sensed electrical activity (e.g., an electrogram (EGM)) of the myocardial tissue proximate thereto.

Additionally, or alternatively to the circumferentially spaced microelectrodes 26, the elongated member 12 may include one or more forward facing microelectrodes 26 (not shown). The forward facing microelectrodes 26 may be generally centrally located within the ablation electrode 24 and/or at an end of a tip of the elongated member 12.

The microelectrodes 26 may be operatively coupled to the processor 16 and the generated output signals from the microelectrodes 26 may be sent to the processor 16 for processing in one or more manners discussed herein and/or for processing in other manners. Illustratively, analyzing an EGM reading of the generated output signals may at least partially form the basis of a contact assessment and/or the basis of determining a level of force applied by the elongated member 12 to the target tissue.

The ablation electrode 24 may be any length and may have any number of microelectrodes 26 positioned therein and spaced circumferentially and/or longitudinally thereabout. In some instances, the ablation electrode 24 may have a length of between one (1) mm and twenty (20) mm, three (3) mm and seventeen (17) mm, or six (6) mm and fourteen (14) mm. In one illustrative example, the tissue ablation electrode 24 may have an axial length of about eight (8) mm.

The plurality of microelectrodes 26 within the ablation electrode 24 may be spaced at any interval about the circumference of the ablation electrode 24. In one example, the ablation electrode 24 may include at least three microelectrodes 26 equally or otherwise spaced about the circumference of the ablation electrode 24 and at the same or different longitudinal positions along the longitudinal axis thereof. In instances when the microelectrodes 26 are equally circumferentially spaced and at the same longitudinal position, as well as in other instances, the three microelectrodes 26 may be configured to form the first bipolar microelectrode pair 28a, the second bipolar microelectrode pair 28b, and the third bipolar microelectrode pair 28c.

The ablation electrode 24 may have an exterior wall that at least partially defines an open interior region (not shown). The exterior wall may include one or more openings for accommodating a microelectrode 26 in each opening. Additionally, or alternatively, the ablation electrode 24 may include one or more irrigation ports (not shown). Illustratively, the irrigation ports, when present, may be in fluid communication with an external irrigation fluid reservoir and pump which may be used to supply fluid to tissue to be or being mapped and/or ablated.

Illustrative catheters that may be used as the elongated member 12 may include, among other ablation and/or mapping catheters, those described in U.S. patent application Ser. No. 12/056,210 filed on Mar. 26, 2008, and entitled HIGH RESOLUTION ELECTROPHYSIOLOGY CATHETER, and U.S. Pat. No. 8,414,579 filed on Jun. 23, 2010, entitled MAP AND ABLATE OPEN IRRIGATED HYBRID CATHETER, which are both hereby incorporated by reference in their entireties for all purposes. Alternatively, or in addition, catheters that may be used as the elongated member 12 may include, among other ablation and/or mapping catheters, those described in U.S. Pat. No. 5,647,870 filed on Jan. 16, 1996, as a continuation of U.S. Ser. No. 206,414, filed Mar. 4, 1994 as a continuation-in-part of U.S. Ser. No. 33,640, filed Mar. 16, 1993, entitled MULTIPLE ELECTRODE SUPPORT STRUCTURES, U.S. Pat. No. 6,647,281 filed on Apr. 6, 2001, entitled EXPANDABLE DIAGNOSTIC OR THERAPEUTIC APPARATUS AND SYSTEM FOR INTRODUCING THE SAME INTO THE BODY, and U.S. Pat. No. 8,128,617 filed on May 27, 2008, entitled ELECTRICAL MAPPING AND CRYO ABLATING WITH A BALLOON CATHETER, where are all hereby incorporated by reference in their entireties for all purposes.

In some illustrative instances, the processor 16 may be capable of detecting, processing and/or recording or may be configured to detect, process, and/or record electrical signals (e.g., electrograms (EGMs)) within the heart via the elongated member 12. Based on the detected, processed, and/or recorded electrical signals, a physician may be able to identify specific target tissue sites within the heart, and may be able to ensure any arrhythmia causing substrates have been electrically isolated by ablative treatment from the RF ablation system 10.

The processor 16 may be capable of processing or may be configured to process the output signals from the microelectrodes 26 and/or the ring electrodes 22. Based on the processed output signals from the microelectrodes 26 and/or the ring electrodes 22, the processor 16 may generate an output to a display (not shown) for use by a physicician or other user.

In some instances, a display may include various static and/or dynamic information related to the use of the RF ablation system 10. In one example, the display may include an indicator with electrocardiograms (ECG) information, which may be analyzed by the processor 16 and/or the user to determine the existence and/or location of arrhythmia substrates within the heart and/or to determine the location of the elongated member 12 within the heart. Illustratively, the output of the processor 16 may be used to provide, via the display, an indication to the clinician about a characteristic of the elongated member 12 and/or the myocardial tissue interacted with and/or being mapped.

The RF generator 14 of the RF ablation system 10 may be capable of delivering and/or may be configured to deliver ablation energy to the elongated member 12 in a controlled manner in order to ablate target tissue sites identified by processor 16. Ablation of tissue within the heart is well known in the art, and thus for purposes of brevity, the RF generator 14 will not be described in further detail. Further details regarding RF generators are provided in U.S. Pat. No. 5,383,874 filed Nov. 13, 1992, and entitled SYSTEMS FOR IDENTIFYING CATHETERS AND MONITORING THEIR USE, which is hereby incorporated by reference in its entirety for any purpose. Although the processor 16 and RF generator 14 may be shown as discrete components, these components or features of components, may be incorporated into a single device.

In addition to or as an alternative to applying ablation energy received from the RF ablation generator 14 to a target tissue and/or mapping a target tissue, the elongated member 12 may be utilized to perform various diagnostic functions to assist the physician in ablation and/or mapping treatments. In one example, the elongated member 12 may be used to ablate cardiac arrhythmias, and at the same time provide real-time assessment of a lesion formed during ablation (e.g., during RF ablation). Real-time assessment of the lesion may involve one or more of monitoring surface and/or tissue temperature at or around the lesion, reduction in an electrocardiogram signal, a drop in impedance, direct and/or surface visualization of the lesion site, and imaging of a tissue site (e.g., using computed tomography, magnetic resonance imaging, ultrasound, etc.). Additionally, or alternatively, the presence of microelectrodes 26 at or about the ablation electrode 24 and/or within the tip (e.g., distal tip) of the elongated member 12 may facilitate allowing a physician to locate and/or position the ablation electrode 24 at a desired treatment site, to determine the position and/or orientation of the tissue ablation electrode relative to the tissue that is to be ablated or relative to any other feature, and/or to determine a level of contact force between the elongate member and a target tissue.

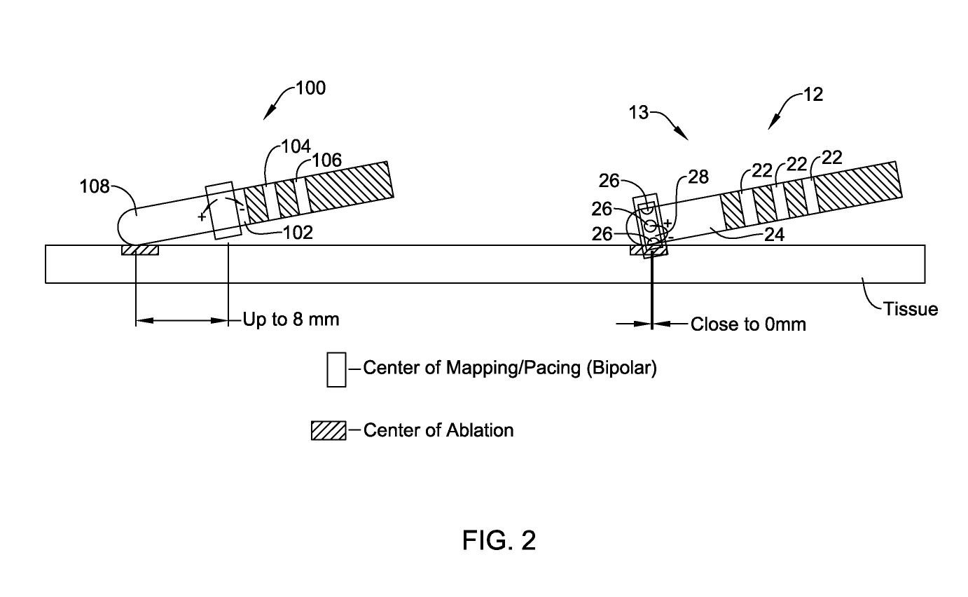

The positioning of electrodes of the elongated member 12 as compared to the positioning of electrodes in a conventional ablation catheter 100 is shown in FIG. 2. FIG. 2 is a schematic illustration showing a conventional ablation catheter 100 (e.g., an ablation catheter lacking any microelectrodes within the tissue ablation electrode) on the left and the elongated member 12 on the right. For cardiac mapping, the conventional ablation catheter 100 relies on conventional ring electrodes 102, 104, 106 disposed a distance from the ablation electrode 108 (e.g., a distance up to or greater than 8 mm). Such positioning of the ring electrodes 102, 104, 106 may result in a large distance between the center of the mapping and the center of the ablation. The elongated member 12, in contrast, may include the microelectrodes 26 in or on the ablation electrode 24 to allow the center of mapping to be in substantially the same location as the center of the ablation, which may facilitate a more accurate understanding of the positioning of the ablation electrode 24 about a target tissue.

### RF Ablation System Analyses

The size and/or location of the microelectrodes 26 within the ablation electrode 24 may allow for the identification of electrophysiologic electrogram (EGM) signatures that may define when the tip (e.g., distal tip) of the elongated member 12 is in contact with the tissue and/or the extent of the contact between the tip of the elongated member 12 and a target tissue (e.g., cardiac tissue, venous tissue, arterial tissue, etc.). The proximity of the microelectrodes 26 to the tip of the elongated member 12, for example, may enable the microelectrodes 26 to detect transient local ischemia that may occur when the tip of the elongated member 12 is pressed into cardiac muscle. In some instances, the local ischemia may be generated from compression of the tissue, which may cause the local cellular conduction to form distinct Monophasic Action Potentials (MAPs) that can be observed from EGM readings or signatures. Illustratively, these MAPs may have a very distinct morphology when either recorded alone or when recorded as part of a multiphasic complex in an EGM reading or signature.

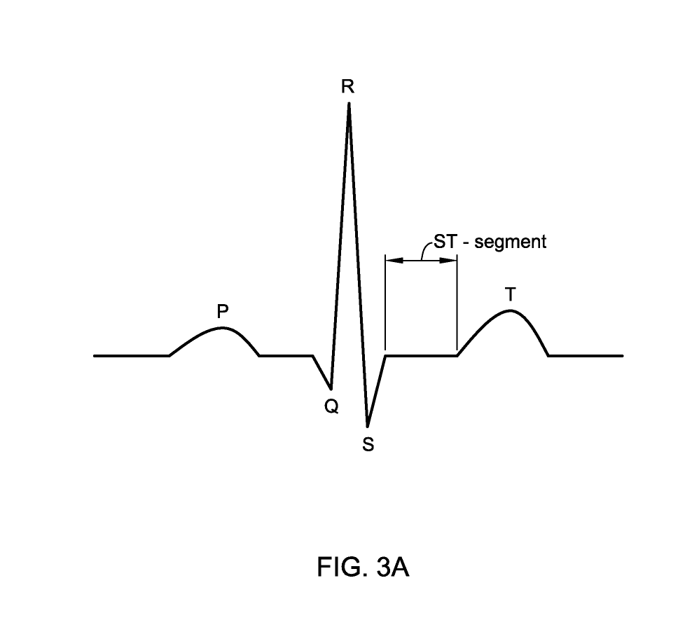

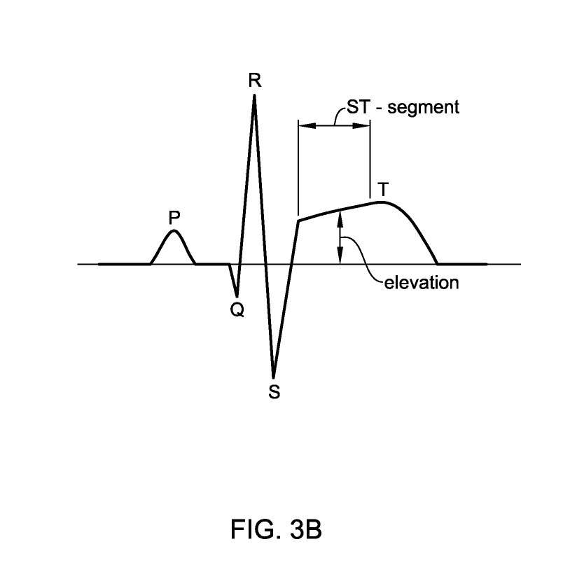

The microelectrodes 26 may utilize a multiphasic bi-polar recording modality which may generate a common P-QRS-T wave EGM. The ST segment of the P-QRS-T wave EGM, which connects the QRS complex with the T-wave, may correspond to a period of ventricle systolic repolarization when the cardiac muscle is contracted. Subsequent relaxation may occur during the diastolic repolarization phase. Illustratively, the normal course of the ST segment, as shown in FIG. 3A, may reflect a certain sequence of muscular layers undergoing repolarization and certain timing of this activity. For example, one may expect the ST segment to follow a pattern when undergoing repolarization, such as a substantially level pattern, as shown in P-QRS-T wave of FIG. 3A.

When the cardiac muscle is damaged and/or undergoes a pathological process (e.g., an injury or ischemia), its contractile and/or electrical properties may change. In some instances the changed contractile and/or changed electrical properties may lead to early repolarization or premature ending of the systole. FIG. 3B depicts a P-QRS-T wave with an elevated ST segment relative to a typical ST segment (as shown in FIG. 3A), where the elevated ST segment may be caused by contact between the tip of the elongated member 12 and cardiac tissue and/or force from the tip of the elongated member 12 to cardiac tissue.

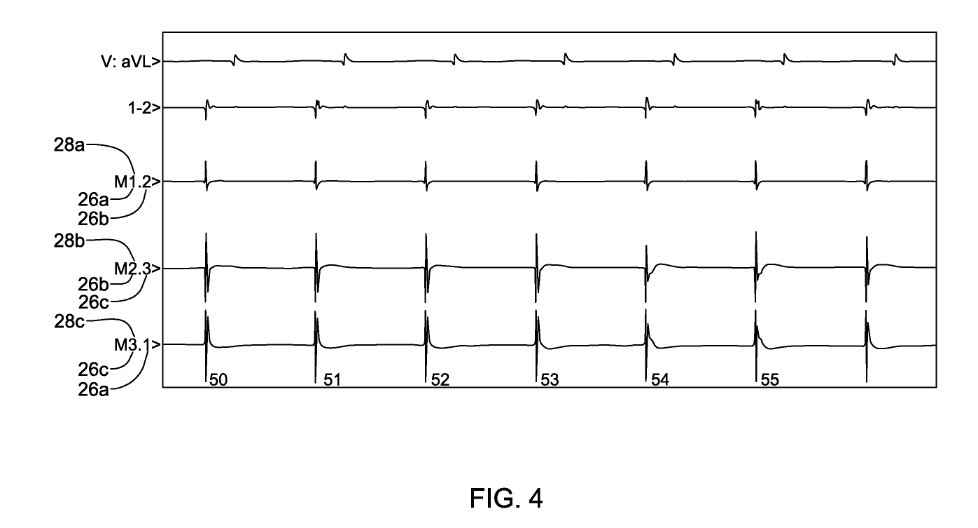

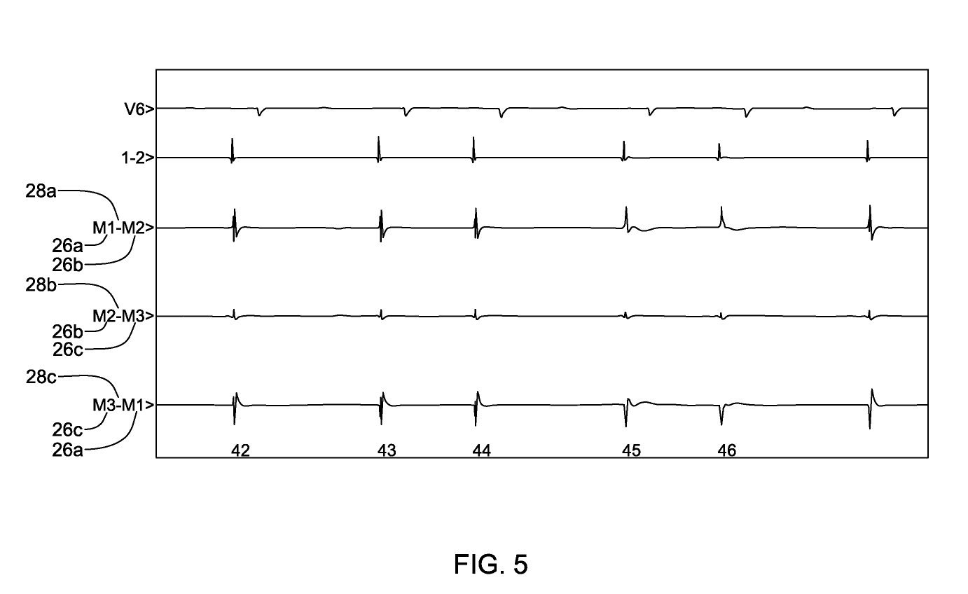

As discussed, the microelectrodes 26 of the elongated member 12 may provide feedback on electrode contact and/or tip electrode orientation within the heart. FIGS. 4 and 5 are schematic illustrations of EGM signals with amplitudes of cardiac electrical signals sensed by microelectrodes 26 and ring electrodes 22 of the elongated member 12. The data depicted in FIGS. 4 and 5 may be used to implement a method for determining electrode contact and/or the orientation of the catheter tip.

Illustratively, in FIGS. 4 and 5, an elongated member 12 having three microelectrodes 26 (e.g., a first microelectrode 26a, a second microelectrode 26b, and a third microelectrode 26c) distributed about the circumference of the tissue ablation electrode 24, such that the circumferentially spaced (e.g., equally circumferentially spaced or otherwise spaced) microelectrodes may form respective bipolar microelectrode pairs 28 of adjacent microelectrodes 26 (e.g., a first bipolar microelectrode pair 28a of the first microelectrode 26a and the second microelectrode 26b, a second bipolar microelectrode pair 28b of the second microelectrode 26b and the third microelectrode 26c, and a third bipolar microelectrode pair 28c of the third microelectrode 26c and the first microelectrode 26a). Illustratively, an EGM signal of one or more (e.g., each) bipolar microelectrode pairs 28 may form the basis of a contact assessment and/or an algorithm for predicting lesion formation.

When the tip of the elongated member 12 is in contact with a target tissue and a level of force (e.g., at least a minor level of force or at least some level of force) is exerted from the elongated member 12 to the cardiac tissue, the ST segment of the P-QRS-T wave may be elevated and apparent in the EGMs obtained from the microelectrodes 26 (e.g., an elevation of an ST segment may be proportional to the level of force exerted on the cardiac tissue by the elongated member 12). In some instances, from the bipolar microelectrode pairs 28 of the microelectrodes 26 arranged circumferentially about the distal tip of the elongated member 12, the orientation of the ablation electrode 24 may be determined based at least in part on the apparent elevated ST segments.

A specific bipolar microelectrode pair 28 or a specific microelectrode 26 of the microelectrodes 26 circumferentially spaced about the distal tip of the elongated member 12 in best contact with cardiac tissue may be determined from the EGMs of the bipolar microelectrode pairs 28. For example, as shown in FIG. 4, P-QRS-T waves may be displayed for each bipolar microelectrode pair 28 (e.g., a first pair 28a between a first microelectrode 26a and a second microelectrode 26b, a second pair 28b between the second microelectrode 26b and a third microelectrode 26c, and a third pair 28c between the third microelectrode 26c and the first microelectrode 26a). In the example, the common microelectrode 26 of adjacent EGMs having elevated ST segments may be the microelectrode 26 that is in best contact with cardiac tissue, as determined by the processor 16 or otherwise determined. Illustratively, as shown in FIG. 4, the second bipolar microelectrode pair 28b and the third bipolar microelectrode pair 28c both have elevated ST segments, from which it may be determined that the common microelectrode 26, the third microelectrode 26c, may have the best or greatest contact of the microelectrodes 26 of the elongated member with cardiac tissue.

Alternatively, or in addition, when the bipolar microelectrodes pairs 28 are positioned circumferentially around the distal tip of the elongated member 12, an apparent change in polarity may be detected in the ST segments of the bipolar microelectrode pairs 28 including the microelectrode 26 in best contact with cardiac tissue. The change in polarity may be due to wave fronts passing through or over (e.g., coming and going) the microelectrode 26 that is in greatest contact with the cardiac tissue. For example, as shown in FIG. 4, the polarity of the second bipolar microelectrode pair 28b is positive and the polarity of the third bipolar microelectrode pair 28c is negative. This pattern in the EGMs of the bipolar microelectrode pairs 28 may be indicative of the common microelectrode 26 (e.g., the third microelectrode 26c in FIG. 4) having the best contact with the cardiac tissue of the circumferentially spaced microelectrodes 26 and/or indicative of rapid lesion formation on the cardiac tissue from the common microelectrode 26 of the bipolar microelectrode pairs 28. In some instances, the common microelectrode 26 may be referred to as the microelectrode 26 off which the bipolar microelectrodes pairs 28 are centered.

In one illustrative example of determining the orientation of the ablation electrode 24 of the elongated member 12 with the processor 16 or otherwise, the bipolar microelectrode pair 28 in greatest contact with the target tissue may be determined from the P-QRS-T wave with the highest amplitude (e.g., highest voltage amplitude) and/or highest frequency spectra. Then, the specific microelectrode 26 in best contact with the target tissue may be determined by determining the common microelectrode 26 of the bipolar microelectrode pairs 28 having the EGMs with the highest ST segment elevation.

Additional discussion of frequency and/or amplitude analyses of P-QRS-T waves of EGM readings is found in U.S. Patent Provisional Application Ser. No. 61/955,087, filed on Mar. 18, 2014, entitled ELECTROPHYSIOLOGY SYSTEM and having Attorney Docket No. 1001.3506100 and in U.S. Patent Application Serial No. ______, filed on Mar. 18, 2015, entitled ELECTROPHYSIOLOGY SYSTEM and having Attorney Docket No. 1001.3506101, which is hereby incorporated by reference in its entirety for all purposes.

In some cases and in addition to or as an alternative to using the elevation of ST segments to determine the orientation of the elongated member 12 with respect to a target tissue, the elevation of the ST segment may be analyzed to determine a level or amount of contact force between the distal portion 13 of the elongated member 12 and a target tissue, as the ST segment elevation may be proportional to the degree/level of contact/force between the distal tip of the elongated member 12 and the target tissue. For example, light contact between the distal tip of the elongated member 12 and the cardiac tissue may produce little or no elevation of the ST segment and significant contact (e.g., contact more significant than the light contact) between the distal tip of the elongated member 12 and the cardiac tissue may produce significant ST segment elevation (e.g., more elevation of the ST segment than is evident when there is light contact between the distal tip of the elongated member 12 and the cardiac tissue). Elevations of ST segments of sequential P-QRS-T waves from one or more of the bipolar microelectrode pairs 28 may be analyzed to determine changes in levels of force applied by the distal portion 13 of the elongated member 12 on a target tissue over time. Additionally, or alternatively, determining a level or amount of contact force between the distal portion 13 of the elongated member 12 and a tissue may include analyzing the elevation of one or more of the ST segments of the EGM readings of the bipolar microelectrode pairs 28 that include the common microelectrode 26 of first and second bipolar microelectrode pairs 28 having positive and negative polarities, respectively. Note, ST segment elevation is measured by determining the absolute value of the elevation of the ST segment from a center line.

In some instances of determining a level of force between the elongated member 12 and a target tissue, the elevations of ST segments of the EGM signals may be compared to one or more threshold levels. For example, if the elevation of an ST segment exceeds a first threshold level, the level of force between the elongated member 12 and the target tissue may be a first level of force (e.g., a scaled level of force, a level of force indicating contact, a level of force indicating no contact, etc.). In the example, if the elevation of an ST segment is at or below a second threshold level, the level of force between the elongated member 12 and the target tissue may be a second level of force (e.g., a scaled level of force, a level of force indicating contact, a level of force indicating no contact, etc.). In some cases, the second level of force may be less than the first level of force. Illustratively, the first threshold level and the second threshold level in the example may be the same or different threshold levels. If the first threshold level and the second threshold level are different threshold levels, an elevation of an ST segment falling between the threshold levels may indicate a third level of force between the elongated member 12 and the target tissue, where the third level of force is between the first level of force and the second level of force. Such comparative analysis may be extrapolated out using more than two threshold levels and/or different threshold levels relative to one another, as desired.

FIG. 5 is a schematic image of a further set of P-QRS-T wave EGMs including P-QRS-T EGMs of the bipolar microelectrode pairs 28 from which the orientation of the elongated member 12 with respect to a tissue may be determined. As can be seen from FIG. 5, the first bipolar microelectrode pair 28a and the third bipolar microelectrode pair 28c have elevated ST segments and opposite polarities. As a result, it may be determined (e.g., by the processor) that the first microelectrode 26a (e.g., the common microelectrode of the bipolar microelectrode pairs 28 with elevated ST segments and/or adjacent opposite polarities) is the microelectrode 26 of the elongated member 12 that is in best or greatest contact with the cardiac tissue. By knowing the microelectrode 26 that is in best or greatest contact with the tissue, the orientation of the elongated member 12 with respect to the tissue is known and/or can be determined based on knowledge of the position of the microelectrodes 26 on the elongated member 12.

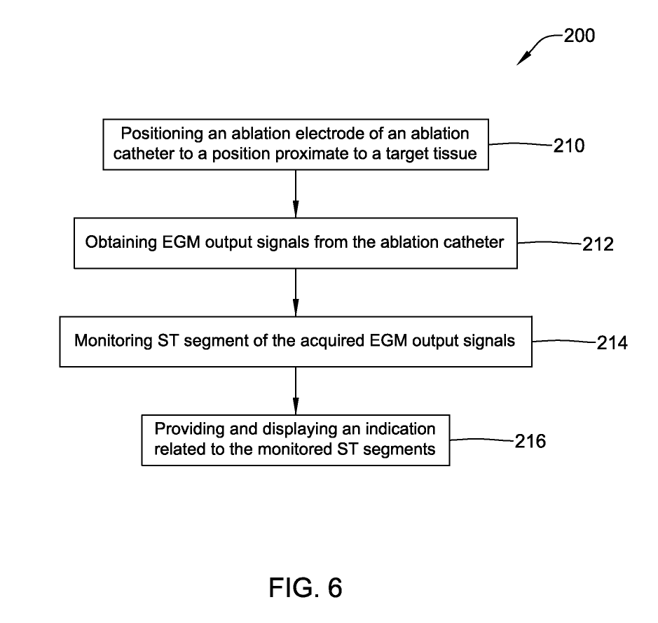

FIG. 6 is a flow chart illustrating a method 200 for assessing a characteristic (e.g., tissue contact, orientation, or the degree/level of force at the tissue contact) of the ablation electrode 24 of the elongated member 12 with the RF ablation system 10. In some instances, one or more analysis features of the method 200 may be performed with the processor 16 in real time (e.g., while positioning the elongated member 12, while performing an ablating procedure, while performing a mapping procedure, and/or while performing any other action or no action), where RF ablation system 10 may include and/or utilizes memory capable of and/or configured to store information (e.g., data, etc.) and computer readable instructions and/or a processor capable of and/or configured to process the stored information and stored computer readable instructions, among processing other data.

Illustratively, the method 200 may include positioning (e.g., advancing, etc.) 210 the distal portion 13 of the elongated member 12 intravascularly to a location proximate a target tissue (e.g., myocardial tissue to be mapped and/or ablated). Before, at the time of, or after the elongated member 12 reaches a target tissue, the ablation system 10 may obtain (e.g., acquire, etc.) 212 EGM output signals or readings from the bipolar microelectrode pairs 28 and/or a bipolar pair between the ablation electrode 24 and the ring electrodes 22. Once the EGM signals have been obtained, an elevation of the ST segment of the P-QRS-T EGMs from each of the EGM signals of the bipolar microelectrode pairs 28 may be monitored 214 (e.g., compared relative to one another, compared relative one or more thresholds, compared over time, and/or monitored in any other manner). Based on the comparisons of the elevation of the ST segments of the P-QRS-T EGMs of the output signal of the bipolar microelectrode pairs 28, an indicator may be activated and in some cases displayed 26 on a display, where the indicator may indicate a condition of the tissue adjacent the ablation electrode 24, a type of the tissue adjacent the ablation electrode 24, an orientation of the elongated member 12 relative to the tissue, contact between the elongated member 12 and tissue, contact force applied to tissue from the elongated member 12, and/or other information related to the use of the elongated member 12 and/or tissue.

In one illustrative example, the displayed condition may include an indication (e.g., one or more of visual, audio, or physical indication) of the proximity of the elongated member 12 to a target tissue (e.g., a myocardial tissue). Example indications may include, but are not limited to, light indications adjacent a bipolar microelectrode pair 28 designation, colored light indications associated with particular bipolar microelectrode pairs 28, text indications, vibration indications, time varied indications, audible indications, any combinations thereof, and/or any other indication.

Indications of proximity may include an indication that the ablation electrode 24 is in contact with the target tissue if the difference between the elevation of any one of the ST segments of the EGM signals from the bipolar microelectrode pairs 28 and any other of the ST segments of the EGM signals from another of the bipolar microelectrode pairs 28 exceeds a predetermined threshold. Alternatively, or in addition, the indication of proximity may include an indication that the ablation electrode 24 is not in contact with the target tissue if the difference between the elevation of any one of the ST segments of the EGM signal from the bipolar microelectrode pairs 28 and any other of the ST segments of the EGM signals from another of the bipolar microelectrode pairs 28 does not exceed a predetermined threshold.

Illustratively, the predetermined threshold for determining the ablation electrode 24 is in contact with a target tissue and the predetermined threshold for determining the ablation electrode 24 is not in contact with a target tissue may be the same or different threshold. If the predetermined thresholds for determining the ablation electrode 24 is in contact with a target tissue or is not in contact with a target tissue are different, an indication may be displayed indicating that no determination with respect to contact between the ablation electrode 24 and a target tissue can be made when a difference in amplitudes of an ST segment of any one of the EGM signals of the bipolar microelectrode pairs 28 and any other of the EGM signals of the bipolar microelectrode pairs 28.

In some illustrative instances, the microelectrodes 26 (and as a result the bipolar microelectrode pairs 28) each may have a known position with respect to the ablation electrode 24 and/or the other microelectrodes 26. From these known positions of the microelectrodes 26, the method 200 may include displaying an indication of the orientation of the ablation electrode 24 relative to a target tissue, as discussed above, based at least partially on comparisons of ST segment elevations of the EGM output signals of the bipolar microelectrode pairs 28 with respect to one another, with respect to a set threshold, with respect to an equation/algorithm in which the ST segment elevations may be entered, and/or with respect to other parameters. In some instances, the displayed indication may be a virtual image of the elongated member 12 oriented with respect to a target in the manner sensed through the microelectrodes 26, an indication of which bipolar microelectrode pair 28 is in the best or greatest contact with a target tissue, an indication of which microelectrode 26 is in the best or greatest contact with a target tissue, and/or any other indication of a characteristic related to the interaction between the elongated member 12 and/or the target tissue.

As referred to above, the amount of force the elongated member exerts on a tissue may be determined from the elevations of the ST segment of the EGMs. From the relationship between the ST segment elevation and the force applied to a tissue by the elongated member 12, the method 200 may include displaying an indication of the amount of force the elongated member 12 is applying to a tissue in real time (e.g., while positioning the ablation electrode 24 or other electrode about a tissue) based on comparisons of ST segment elevations of the EGM output signals of the bipolar microelectrode pairs 28 with respect to sequential P-QRS-T waves from respective bipolar microelectrode pairs 28, with respect to P-QRS-T waves of other bipolar microelectrode pairs 28, with respect to one or more thresholds, with respect to an equation/algorithm in which the ST segment elevations may be entered, and/or with respect to other parameters. In some instances, the displayed indication may be changes in a color of a distal end of a virtual elongated member displayed on a display, a numerical value, a set of lights, any combination thereof, and/or any other indication.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

### ADDITIONAL EXAMPLES

In a first example, a system may include: an elongated member having a distal portion, the distal portion of the elongated member including one or more electrodes; a radio frequency generator operatively coupled to one or more of the electrodes for generating ablation energy to be conveyed to the coupled one or more electrodes; a processor operatively coupled to one or more of the electrodes, the processor is capable of: obtaining output signals from one or more of the electrodes, one or more of the output signals including an electrogram (EGM) reading; and monitoring an elevation of an ST segment of one or more of the EGM readings.

In a second example, the system of example one, wherein the processor is capable of determining a level of force between the elongated member and a target tissue, where the level of force is proportional to one or more of the monitored elevations of ST segments.

In a third example, the system of either one of example one or example two, further including: an indicator in communication with the processor, the indicator is capable of indicating a characteristic related to one or more of the monitored elevations of ST segments.

In a fourth example, the system of any one of examples one through three, wherein the one or more electrodes of the distal portion of the elongated member includes: a tissue ablation electrode configured to apply ablation energy to the target tissue; and a plurality of microelectrodes distributed about the tissue ablation electrode and electrically isolated therefrom, the plurality of microelectrodes defining a plurality of bipolar microelectrode pairs, each bipolar microelectrode pair configured to generate an output signal.

In a fifth example, the system of example four, wherein the plurality of microelectrodes include three microelectrodes defining a first bipolar microelectrode pair, a second bipolar microelectrode pair, and a third bipolar microelectrode pair.

In a sixth example, the system of either one of example four or example five, wherein two or more of the plurality of microelectrodes are disposed at a same longitudinal position along the tissue ablation electrode.

In a seventh example, the system of any one of examples four through six, wherein the processor is capable of determining which bipolar microelectrode pair is in best contact with a target tissue.

In an eight example, the system of example seven, wherein processor is capable of determining which bipolar microelectrode pair is in best contact with the target tissue from one or more of a voltage related measure of one or more EGM reading and a frequency related measure of one or more EGM reading.

In a ninth example, the system of any one of examples one through eight, wherein the processor is capable of determining which electrode is in greatest contact with a target tissue.

In a tenth example, the system of example nine, wherein: the one or more electrodes includes microelectrodes forming a plurality of bipolar microelectrode pairs; and determining which electrode is in greatest contact with a target tissue includes identifying a common microelectrode of a first bipolar microelectrode pair having a positive elevated ST segment and of a second bipolar microelectrode pair having a negative elevated ST segment.

In an eleventh example, the system of example ten, wherein a determined level of force between the elongated member and a target tissue is proportional to the elevation of one or more of the ST segments of the EGM readings of the bipolar microelectrode pairs that include the common microelectrode.

In a twelfth example, the system of any one of examples one through eleven, wherein: the elongated member further includes a handle having a control element for manipulation by a user, and the distal portion of the elongated member is deflectable upon manipulation of the control element.

In a thirteenth example, a method including: positioning a distal portion of an elongated member at a location proximate a target tissue, the distal portion of the elongated member including one or more electrodes; obtaining an output signal from one or more of the electrodes, one or more of the output signals includes an electrogram (EGM) reading; and monitoring an elevation of an ST segment of one or more of the EGM readings.

In a fourteenth example, the method of example thirteen, further including: determining which electrode is in greatest contact with the target tissue.

In a fifteenth example, the method of either one of example thirteen or example fourteen, wherein: the one or more electrodes include microelectrodes forming a plurality of bipolar microelectrode pairs; and determining which electrode is in greatest contact with the target tissue is determined by a common microelectrode of a first bipolar microelectrode pair having a positive elevated ST segment and of a second bipolar microelectrode pair having a negative elevated ST segment.

In a sixteenth example, the method of any one of examples thirteen through fifteen, further including: determining a level of force between the elongated member and the target tissue, where the level of force is proportional to one or more of the elevations of ST segments.

In a seventeenth example, the method of example sixteen, further including: comparing the elevations of ST segments to one or more threshold levels.

In an eighteenth example, the method of either one of example sixteen and example seventeen, wherein determining a level of force between the elongated member and the target tissue includes: determining the level of force is a first level of force if one or more of the elevations of ST segments exceeds a first threshold level; and determining the level of force is a second level of force if one or more of the elevations of ST segments is at or below a second threshold level.

In a nineteenth example, a system including: an elongated member having a distal portion, the distal portion of the elongated member including: a tissue ablation electrode configured to apply ablation energy to the target tissue; a plurality of microelectrodes distributed about the tissue ablation electrode and electrically isolated therefrom, the plurality of microelectrodes defining a plurality of biopolar microelectrode pairs, each bipolar microelectrode pair configured to generate an output signal; a radio frequency generator operatively coupled to the tissue ablation electrode for generating ablation energy to be conveyed to the tissue ablation electrode; a mapping processor operatively coupled to one or more of the electrodes, the mapping processor is capable of: obtaining output signals from one or more of the electrodes, one or more of the output signals include an electrogram (EGM) reading from one of the bipolar microelectrode pairs; comparing an elevation of an ST segment of the EGM reading from a bipolar microelectrode pair to elevations of ST segments of the EGM readings of the other bipolar microelectrode pairs to determine a level of contact force between the distal portion of the elongated member and the target tissue; and an indicator in communication with the mapping processor for indicating the level of contact force between the distal portion of the elongated member and the target tissue.

In a twentieth example, the system of example nineteen, wherein the mapping processor is capable of: determining a first level of contact force if a difference between the elevation of an ST segment of any one of the EGM readings of the bipolar microelectrode pairs and the elevation of an ST segment of any other of the EGM readings of the bipolar microelectrode pairs exceeds a first threshold; and determining a second level of contact force if a difference between the elevation of an ST segment of any one of the EGM readings of the bipolar microelectrode pairs and an elevation of an ST segment of any other of the EGM readings of the bipolar microelectrode pairs is less than a second threshold.

What is claimed is:

1. A system comprising:
an elongated member having a distal portion, the distal portion of the elongated member comprising one or more electrodes;
a radio frequency generator operatively coupled to one or more of the electrodes for generating ablation energy to be conveyed to the coupled one or more electrodes;
a processor operatively coupled to one or more of the electrodes, the processor is capable of:
obtaining output signals from one or more of the electrodes, one or more of the output signals comprises an electrogram (EGM) reading; and
monitoring an elevation of an ST segment of one or more of the EGM readings.

2. The system of claim 1, wherein the processor is capable of determining a level of force between the elongated member and a target tissue, where the level of force is proportional to one or more of the monitored elevations of ST segments.

3. The system of claim 1, further comprising:
an indicator in communication with the processor, the indicator is capable of indicating a characteristic related to one or more of the monitored elevations of ST segments.

4. The system of claim 1, wherein the one or more electrodes of the distal portion of the elongated member comprises:
a tissue ablation electrode configured to apply ablation energy to a target tissue; and
a plurality of microelectrodes distributed about the tissue ablation electrode and electrically isolated therefrom, the plurality of microelectrodes defining a plurality of bipolar microelectrode pairs, each bipolar microelectrode pair configured to generate an output signal.

5. The system of claim 4, wherein the plurality of microelectrodes include three microelectrodes defining a first bipolar microelectrode pair, a second bipolar microelectrode pair, and a third bipolar microelectrode pair.

6. The system of claim 4, wherein two or more of the plurality of microelectrodes are disposed at a same longitudinal position along the tissue ablation electrode.

7. The system of claim 4, wherein the processor is capable of determining which bipolar microelectrode pair is in best contact with a target tissue.

8. The system of claim 7, wherein processor is capable of determining which bipolar microelectrode pair is in best contact with the target tissue from one or more of a voltage related measure of one or more EGM reading and a frequency related measure of one or more EGM reading.

9. The system of claim 1, wherein the processor is capable of determining which electrode is in greatest contact with a target tissue.

10. The system of claim 9, wherein:
the one or more electrodes include microelectrodes forming a plurality of bipolar microelectrode pairs; and
determining which electrode is in greatest contact with a target tissue includes identifying a common microelectrode of a first bipolar microelectrode pair having a positive elevated ST segment and of a second bipolar microelectrode pair having a negative elevated ST segment.

11. The system of claim 10, wherein a determined level of force between the elongated member and a target tissue is proportional to the elevation of one or more of the ST segments of the EGM readings of the bipolar microelectrode pairs that include the common microelectrode.

12. The system of claim 1, wherein:
the elongated member further comprises a handle having a control element for manipulation by a user, and
the distal portion of the elongated member is deflectable upon manipulation of the control element.

13. A method comprising:
positioning a distal portion of an elongated member at a location proximate a target tissue, the distal portion of the elongated member comprising one or more electrodes;
obtaining an output signal from one or more of the electrodes, one or more of the output signals comprises an electrogram (EGM) reading; and
monitoring an elevation of an ST segment of one or more of the EGM readings.

14. The method of claim 13, further comprising:
determining which electrode is in greatest contact with the target tissue.

15. The method of claim 13, wherein:
the one or more electrodes include microelectrodes forming a plurality of bipolar microelectrode pairs; and
determining which electrode is in greatest contact with the target tissue is determined by a common microelectrode of a first bipolar microelectrode pair having a positive elevated ST segment and of a second bipolar microelectrode pair having a negative elevated ST segment.

16. The method of claim 13, further comprising:
determining a level of force between the elongated member and the target tissue, where the level of force is proportional to one or more of the elevations of ST segments.

17. The method of claim 16, further comprising:
comparing the elevations of ST segments to one or more threshold levels.

18. The method of claim 16, wherein determining a level of force between the elongated member and the target tissue comprises:
determining the level of force is a first level of force if one or more of the elevations of ST segments exceeds a first threshold level; and
determining the level of force is a second level of force if one or more of the elevations of ST segments is at or below a second threshold level.

19. A system comprising:
an elongated member having a distal portion, the distal portion of the elongated member including:
a tissue ablation electrode configured to apply ablation energy to a target tissue;
a plurality of microelectrodes distributed about the tissue ablation electrode and electrically isolated therefrom, the plurality of microelectrodes defining a plurality of biopolar microelectrode pairs, each bipolar microelectrode pair configured to generate an output signal;
a radio frequency generator operatively coupled to the tissue ablation electrode for generating ablation energy to be conveyed to the tissue ablation electrode;
a mapping processor operatively coupled to one or more of the electrodes, the mapping processor is capable of:
obtaining output signals from one or more of the electrodes, one or more of the output signals comprises an electrogram (EGM) reading from one of the bipolar microelectrode pairs;
comparing an elevation of an ST segment of the EGM reading from a bipolar microelectrode pair to elevations of ST segments of the EGM readings of the other bipolar microelectrode pairs to determine a level of contact force between the distal portion of the elongated member and the target tissue; and
an indicator in communication with the mapping processor for indicating the level of contact force between the distal portion of the elongated member and the target tissue.

20. The system of claim 19, wherein the mapping processor is capable of:
determining a first level of contact force if a difference between the elevation of an ST segment of any one of the EGM readings of the bipolar microelectrode pairs and the elevation of an ST segment of any other of the EGM readings of the bipolar microelectrode pairs exceeds a first threshold; and
determining a second level of contact force if a difference between the elevation of an ST segment of any one of the EGM readings of the bipolar microelectrode pairs and an elevation of an ST segment of any other of the EGM readings of the bipolar microelectrode pairs is less than a second threshold.
